(19) United States
(12) Patent Application Publication
(10) Pub. No.: US 2020/0405209 A1
(43) Pub. Date: Dec. 31, 2020

(54) AUTOMATED DETECTION OF COGNITIVE CONDITIONS

(71) Applicant: ROWAN UNIVERSITY, GLASSBORO, NJ (US)

(72) Inventors: DAVID J. LIBON, PHILADELPHIA, PA (US); GANESH BALIGA, PRINCETON, NJ (US); MARY LOUISE E. KERWIN, ASTON, PA (US); RODNEY A. SWENSON, FARGO, ND (US)

(73) Assignee: ROWAN UNIVERSITY, GLASSBORO, NJ (US)

(21) Appl. No.: 16/970,528

(22) PCT Filed: Mar. 1, 2019
(86) PCT No.: PCT/US2019/020365

§ 371 Date: Aug. 17, 2020

Related U.S. Application Data
(60) Provisional application No. 62/637,172, filed on Mar. 1, 2018.

(51) Int. Cl. A61B 5/16; A61B 5/00; G16H 50/20; G16H 40/67

(52) U.S. Cl. CPC A61B 5/162; A61B 5/4803; A61B 5/741; A61B 5/7435; A61B 5/7475; G16H 40/67; A61B 5/4088; A61B 5/0022; A61B 5/6898; G16H 50/20; A61B 5/7282

(57) ABSTRACT

A computer-implemented method for diagnosing a neurological or medical condition of a patient is described herein. In one embodiment, the computer-implemented method may include presenting a set of values via a graphical user interface or an audio speaker, receiving at least a first response from the patient based on the presented set of values, determining at least one time lapse between presenting the set of values and receiving the at least first response, and identifying the neurological or medical condition of the patient based on the at least one time lapse.

### CROSS-REFERENCE TO RELATED APPLICATION(S)

This application claims priority to U.S. Provisional Patent Application Ser. No. 62/637,172, filed Mar. 1, 2018. The entire content of this application is hereby incorporated by reference herein.

### BACKGROUND OF THE INVENTION

Neurological conditions, such as dementia including Alzheimer's disease and related dementia syndromes, as well as prodromal neurological syndromes such as mild cognitive impairment (MCI) conditions, are known to present with impairment in neuropsychological abilities including executive control, episodic memory, language, thinking, and judgment that are greater than typical, age-related declines. However, conditions related to MCI may be difficult to identify or diagnose, particularly when patients are comparatively young and medically healthy.

### SUMMARY

One aspect of the invention provides for a computer-implemented method for automated detection of cognitive conditions as described herein. In one embodiment, the computer-implemented method may include presenting a set of values via a graphical user interface or an audio speaker, receiving at least a first response from the patient based on the presented set of values, determining at least one time lapse between presenting the set of values and receiving the at least first response, and identifying the neurological or medical condition of the patient based on the at least one time lapse.

This aspect of the invention can include a variety of embodiments.

One embodiment may include determining that the at least first response comprises an incorrect or correct response, where identifying the neurological or medical condition is further based on the determined incorrect or correct response. Additionally or alternatively, the set of values further includes a first value and at least one other value.

One embodiment may include receiving a second response from the patient corresponding to the at least one other value, and determining a second time lapse between presenting the set of values and receiving the second response, where identifying the neurological or medical condition of the patient is further based on the second time lapse. Additionally or alternatively, the embodiment may include comparing the first time lapse with the second time lapse, where identifying the neurological or medical condition of the patient is further based on the comparison.

One embodiment may include presenting a set of instructions for responding to the presented set of values in a specified and rearranged order. Additionally or alternatively, the set of instructions are associated with a Backward Digit Span Test (BDST). Additionally or alternatively, the set of instructions are associated with a Philadelphia Pointing Span Test (PPST).

One embodiment may include the at least first response corresponding to reciting the first value. Additionally or alternatively, the at least first response can include an audible response. Additionally or alternatively, the at least first response can include a physical touch on the graphical user interface.

One embodiment may include the neurological or medical condition including a mild cognitive impairment (MCI) and related neurological illness.

One embodiment may include transmitting a first time lapse value and information corresponding to the neurological or medical condition to a storage device or cloud storage entity. Additionally or alternatively, the embodiment may include granting access to the first time lapse value and the information corresponding to the neurological or medical condition from the storage device or the cloud storage entity through a web browser.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system for automated detection of cognitive conditions, according to an embodiment of the claimed invention.

FIG. 2 depicts a mobile device for implementing automated detection of cognitive conditions, according to an embodiment of the claimed invention.

FIG. 3 depicts a workflow process for a reviewing user to access and review the medical condition identification, according to an embodiment of the claimed invention.

FIG. 4 depicts a workflow process for automated detection of cognitive conditions, according to an embodiment of the claimed invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within one standard deviation of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

### Automated Detection of Cognitive Conditions

Neurological or medical conditions, such as mild cognitive impairment (MCI) conditions, may involve issues with executive control, episodic memory, language, thinking, and judgment that are greater than typical, age-related declines. However, conditions related to MCI may be difficult to identify or diagnose, particularly with patients in the age range where cognitive decline is not expected.

The claimed invention discussed herein provides for an automated latency-based system and process for identifying or diagnosing emerging neurological or medical conditions of a patient. The system provides a patient with a series of values that the patient must recite back. In some cases, the series of values is requested in a specified order. The system times the patient in providing responses back corresponding to the series of values. Based on the timing, the system can identify latencies between responses provided, and based on the latencies, may identify or diagnose the patient with a neurological or medical condition. For example, latencies that exceed one standard deviation (above or below) relative to normative values suggest derailed capacity to marshal the necessary neuropsychological resources to cope with test demands and is judged to be diagnostic of neurological/medical conditions such as mild cognitive impairment or a known prodromal condition related to dementia such as Alzheimer's disease.

### Mobile Device

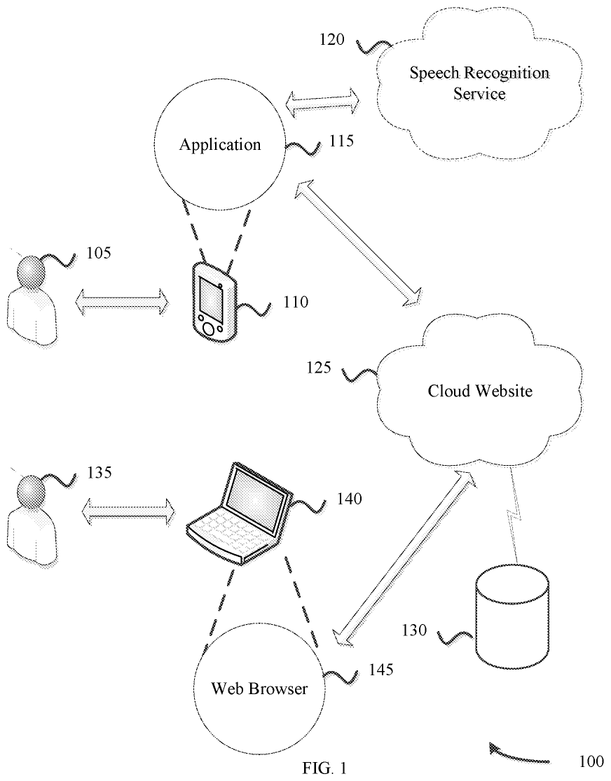

FIG. 1 provides a system for automated detection of cognitive conditions in accordance with an embodiment of the claimed invention. A first user 105 (e.g., a patient) may have access to mobile device 110. The mobile device 110 may be a mobile phone, a personal device assistant, a tablet, a laptop, a computer, a smart watch, etc. The mobile device 110 may include an application 115 that grants the user 105 access to automated detection software. The software may be stored on the mobile device 110, or in some cases on a server, such as server 130.

### Application

The user 105 may open the application 115 and may initiate the automated detection process. The application 115 may provide the user 105, e.g., via a graphical user interface of the mobile device 110 or verbally via a speaker of the mobile device 110, with a set of values, such as a series of number, characters, words, etc. The application 115 may request the user 105 to recite back the set of values to the application 115. Further, the application 115 may request the recitation to be either audible or through touching the graphical user interface (e.g., a touchscreen) of the mobile device 110. In some cases, the application 115 may request the recitation to be conducted in a specified order of the set of values (e.g., “12345” is provided to the user, and the application requests that “54321” is recited back).

The application 115 may receive the responses back from the user 105 and may log the times received for each value. For example, the user 105 may be requested to recite back “321.” The application 115 may log the time that “3” is received, the time when “2” is received, and the time when “1” is received. Based on these logged times, the application 115 may identify time lapses between the received responses. For example, the application 115 may subtract the time from when the user 105 is first presented with the instructions from the time logged for receiving the first response. In another example, the application 115 may subtract the logged time for the first response from the logged time from the second response (e.g., the logged time for “1” subtracted from the logged time from “2”). In another example, the application 115 may subtract the logged time for the first response from the logged time from the third response (e.g., the logged time for “1” subtracted from the logged time from “3”). While the above examples are performed by the application 115, since the application 115 may forward the user 105 to software run on another entity (e.g., server 130), the other entity may perform these functions as well.

In some cases, the application 115 may additionally identify the accuracy of the recitation. For example, in the “321” scenario above, if the user 105 instead recites “312” or “231” when the application requested that the user 105 responds with “321,” the application 115 may log not only the time each response was received, but whether the received response was the correct value. In some cases, the application 115 may store the correct and incorrect responses for later review and/or analysis.

The application 115 may determine a neurological or medical condition (e.g., MCI-related condition, concussion, etc.) of the user 105 based on the determined time lapses. For example, the application 115 may analyze different time lapses, such as total time to completion for the set of values, intra-component latency between different recited values, specific-order recall (e.g., the recitation of the values in a specified, directed order), any-order recall (e.g., the recitation of the values regardless of the order), average time lapse, mean time lapse, median time lapse, etc. These metrics may be analyzed and weighted according to predefined parameters provided for the application (e.g., test-specific, manually provided by a provider/physician, etc.). Based on the metrics, the application 115 may determine that the user 105 exhibits signs of a medical condition such as a MCI-related condition or illness. Again, while the above examples are performed by the application 115, since the application 115 may forward the user 105 to software run on another entity (e.g., server 130), the other entity may perform these functions as well.

### Speech Recognition Service

In some cases, the application 115 may be wirelessly connected to a speech recognition service 120. The speech recognition service 120 may receive data from the application 115 related to audible responses received from the user 105. The speech recognition service 120 may identify the audible response and may log the time the audible response is received from the mobile device 110. Further, the speech recognition service 120 may also identify the content of the audible response. The time log information and the content information may be transmitted to the application 115 for further storage and/or analysis.

### Cloud Website

The application 115 may transmit the information determined from the responses (e.g., the time logs, the accuracy of the responses, the determined latencies, the identified medical conditions, a patient ID, etc.) to the cloud website 125. The application 115 may be in wireless communication with the cloud website 125. Further, the cloud website 125 may be run or managed by the server 130. In some cases, portions of the process performed by the application 115 may instead be performed by the cloud website 125. For example, the application 115 may transmit the response metrics to the cloud website 125, which may then identify a medical condition of the user 105. In some examples, the time logs of the responses are transmitted to the cloud website 125, where the cloud website 125 may then determine the time lapses and subsequently identify a neurological or medical condition.

### Server

As discussed above, the server 130 may be utilized to perform portions of the process that is performed in the above examples by the application 115 and/or the cloud website 125. Additionally, the server 130 may also store the data (e.g., responses, time lapses, identified medical condition, patient ID, audio playback of the responses, etc.) for future access.

### Second Mobile Device

A second user 135 (e.g., a physician) may have access to mobile device 140. The mobile device 140 may be a mobile phone, a personal device assistant, a tablet, a laptop, a computer, a smart watch, etc. The mobile device 140 may include a web browser 145 that grants the user 135 access to the cloud website 125.

The second user 135 may open the web browser 145 on the mobile device 140 any may access the cloud website 125. The cloud website 125 may require authentication (e.g., username and password or other credentials). Once granted access, the second user 135 may request access to the stored data for the first user 105. The cloud website 125 may upload the stored data form the server 130 and may subsequently transmit the stored data to the mobile device 140 for the second user's review. The second user 135 may be able to review the data and the identified neurological or medical conditions. Further, the second user 135 may have authority to revise the identified neurological or medical condition, or instead may verify the identified neurological or medical condition. For example, in the case of the second user 135 being the physician of the first user 105, the physician may be able to verify whether the diagnosis performed by the system 100 is proper.

### Exemplary Tests Performed by System

Various tests that are implemented to determine neurological or medical impairment may be utilized by the system 100. For example, two such exemplary tests that can be implemented are the Backward Digit Span Test (BDST) and the Philadelphia Point Span Test (PPST). The BDST includes a predefined number (e.g., 21) of test trials. For example, the BDST may include 7 trials of 3-span, 7 trials of 4-span, and 7 trials of 5-span digits sequence. Further, the user 105 may be requested to repeat the presented number sequence in a backwards order (e.g., “46975” is correctly recited as “57964”).

The PPST may include 2 subtests: the PPST-digits subtest and the PPST-digit/letter test condition. PPST-digits subtest includes a predefined number (e.g., 15) of test trials. For example, the PPST-digits subtest may include 5 3-span, 5 4-span, and 5 5-span digits sequence. Further, the user 105 may be requested to repeat the presented number sequence in a lowest value to highest value order. The PPST-digit/letter test condition may also include a predefined number (e.g., 15) of test trials. For example, the PPST-digit/letter test condition may include 5 3-span, 5 4-span, and 5 5-span digits/letter sequence. The user 105 may be requested to order the digits first from lowest to highest, followed by letters in alphabetical order (e.g., the system presents “9T46K,” and the correct response is “469KT”). The metrics determined from the responses of the PPST test may allow for the calculation of a neurocognitive index score (NCRI).

### Exemplary Mobile Device

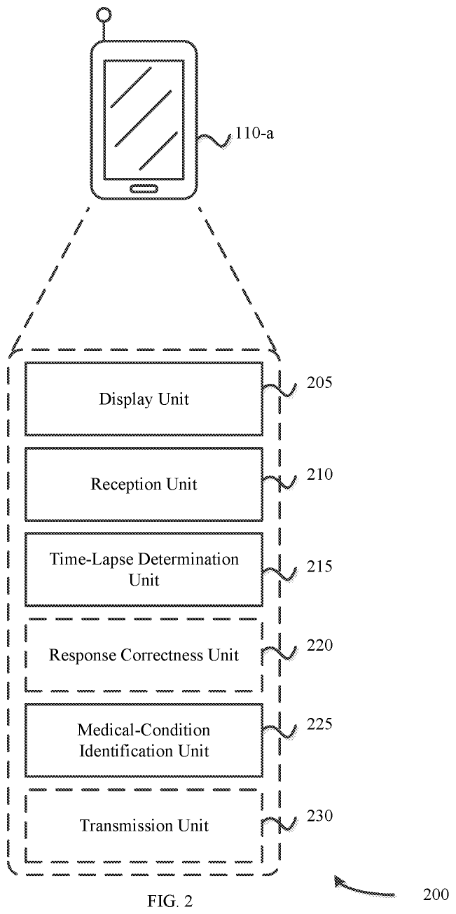

FIG. 2 provides an example of a mobile device 110-a for automated detection of neurological or medical conditions in accordance with an embodiment of the claimed invention. The mobile device 110-a may include a display unit 205, a reception unit 210, a time-lapse determination unit 215, a response correctness unit 220, a medical-condition identification unit 225, and a transmission unit 230.

The display unit 205 may present a set of values for the user 105 to recite, via a graphical user interface or an audio speaker, and corresponding instructions. The display unit 205 may also receive responses from the user 105.

The reception unit 210 may receive audible communications from the user 105.

Additionally or alternatively, the reception unit 120 may receive wireless communications from other electronic devices or entities, such as with cloud website 125.

The time-lapse determination unit 215 may determine time lapses between responses received and/or initiation of the process.

The response correctness unit 220 may determine the accuracy of the responses provided. The accuracy of the responses may be based on the set of instructions presented by the display unit 205.

The neurological/medical-condition identification unit 225 may identify a neurological or medical condition of the user 105 based on the time lapses and in some cases the accuracy of the responses received.

The transmission unit 230 may transmit the findings of the mobile device 110-a to the server 130 (e.g., via cloud website 125). The transmission unit 230 may also be used for wireless communication with other wireless devices and entities.

### Exemplary Physician Workflow

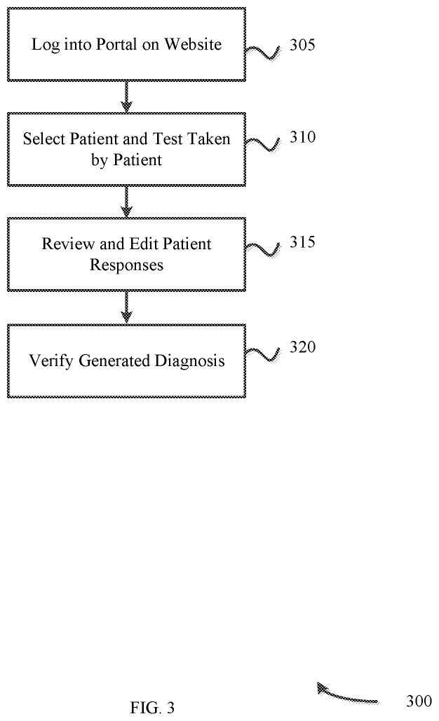

FIG. 3 illustrates a process workflow 300 for a physician according to an embodiment of the claimed invention. At Step 305, a physician may log into a portal of a website, such as cloud website 125. At Step 310, the physician may select a patient, such as user 105, and tests taken by the patient. At Step 315, the physician may review the results of the test, including patient responses, corresponding time lapses, correctness of responses, and identified medical conditions. Additionally, the physician may optionally edit the results of the patient. At Step 320, the physician may verify the diagnosis of the patient.

### Exemplary Patient Workflow

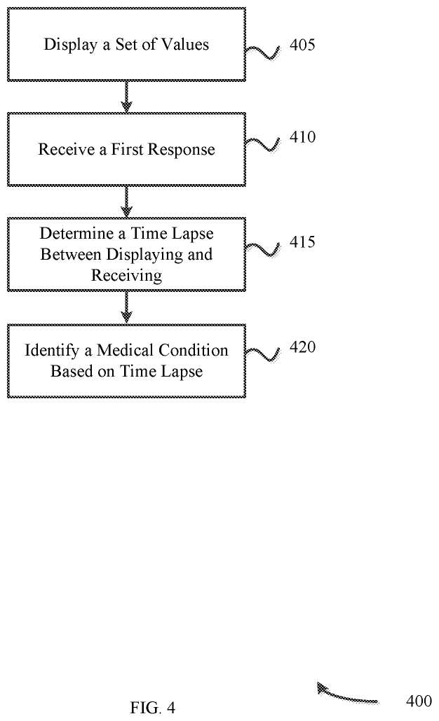

FIG. 4 illustrates a process workflow 400 for automated detection of cognitive conditions according to an embodiment of the claimed invention. The process workflow may be implemented by a mobile device, such as mobile device 110 of FIG. 1. At Step 405, the mobile device may present a set of values. Further, the mobile device may present a set of instructions corresponding to reciting the set of values by a patient (e.g., a user 105). At Step 410, the mobile device may receive a first response from the patient. The first response may correspond to a first value of the set of values. At Step 415, the mobile device may determine a time lapse between presenting the set of values and receiving the response. At Step 420, the mobile device may identify a neurological or medical condition of the patient based on the determined time lapse.

### Software

The above-described steps can be implemented using standard well-known programming techniques. The novelty of the above-described embodiment lies not in the specific programming techniques but in the use of the steps described to achieve the described results. Software programming code which embodies the present invention is typically stored in permanent storage. In a client/server environment, such software programming code may be stored with storage associated with a server. The software programming code may be embodied on any of a variety of known media for use with a data processing system, such as a diskette, or hard drive, or CD ROM. The code may be distributed on such media, or may be distributed to users from the memory or storage of one computer system over a network of some type to other computer systems for use by users of such other systems. The techniques and methods for embodying software program code on physical media and/or distributing software code via networks are well known and will not be further discussed herein.

It will be understood that each element of the illustrations, and combinations of elements in the illustrations, can be implemented by general and/or special purpose hardware-based systems that perform the specified functions or steps, or by combinations of general and/or special-purpose hardware and computer instructions.

These program instructions may be provided to a processor to produce a machine, such that the instructions that execute on the processor create means for implementing the functions specified in the illustrations. The computer program instructions may be executed by a processor to cause a series of operational steps to be performed by the processor to produce a computer-implemented process such that the instructions that execute on the processor provide steps for implementing the functions specified in the illustrations. Accordingly, the figures support combinations of means for performing the specified functions, combinations of steps for performing the specified functions, and program instruction means for performing the specified functions.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method for diagnosing a neurological or medical condition of a patient, the computer-implemented method comprising:
presenting a set of values via a graphical user interface or an audio speaker;
receiving at least a first response from the patient based on the presented set of values;
determining at least one time lapse between presenting the set of values and receiving the at least first response; and
identifying the neurological or medical condition of the patient based on the at least one time lapse.

2. The computer-implemented method of claim 1, further comprising:
determining that the at least first response comprises an incorrect or correct response, wherein identifying the neurological or medical condition is further based on the determined incorrect or correct response.

3. The computer-implemented method of claim 1, wherein the set of values further comprises a first value and at least one other value.

4. The computer-implemented method of claim 3, wherein the at least first response corresponds to reciting the first value.

5. The computer-implemented method of claim 4, further comprising:
receiving a second response from the patient corresponding to the at least one other value; and
determining a second time lapse between presenting the set of values and receiving the second response, wherein identifying the neurological or medical condition of the patient is further based on the second time lapse.

6. The computer-implemented method of claim 5, further comprising:
comparing the at least one time lapse with the second time lapse, wherein identifying the neurological or medical condition of the patient is further based on the comparison.

7. The computer-implemented method of claim 1, further comprising:
presenting a set of instructions for responding to the presented set of values in a specified and rearranged order.

8. The computer-implemented method of claim 7, wherein the set of instructions are associated with a Backward Digit Span Test (BDST).

9. The computer-implemented method of claim 7, wherein the set of instructions are associated with a Philadelphia Pointing Span Test (PPST).

10. The computer-implemented method of claim 1, wherein the at least first response comprises an audible response.

11. The computer-implemented method of claim 1, wherein the at least first response comprises a physical touch on the graphical user interface.

12. The computer-implemented method of claim 1, wherein the neurological or medical condition comprises a mild cognitive impairment (MCI) and related neurological illness.

13. The computer-implemented method of claim 1, further comprising:
transmitting a first time lapse value and information corresponding to the neurological or medical condition to a storage device or cloud storage entity.

14. The computer-implemented method of claim 13, further comprising:
granting access to the first time lapse value and the information corresponding to the neurological or medical condition from the storage device or the cloud storage entity through a web browser.
